(19) United States
(12) Patent Application Publication
(10) Pub. No.: US 2018/0361147 A1
(43) Pub. Date: Dec. 20, 2018

(54) SYSTEMS AND METHODS FOR PROVIDING TEMPORARY INDUCED DYSSYNCHRONY THERAPY

(71) Applicant: PACESETTER, INC., Sylmar, CA (US)

(72) Inventors: Edward Karst, Los Angeles, CA (US); Yelena Nabutovsky, Mountain View, CA (US); Jennifer Rhude, Carbondale, IL (US); Taraneh G. Farazi, San Jose, CA (US)

(21) Appl. No.: 15/628,460

(22) Filed: Jun. 20, 2017

(51) Int. Cl. A61N 1/365; A61N 1/05

(52) U.S. Cl. CPC A61N 1/365; A61N 1/368; A61N 1/056

(57) ABSTRACT

The present disclosure describes systems and methods for providing temporary induced dyssynchrony (TID) therapy. An implantable cardiac device includes a pulse generator coupled to a plurality of electrodes, and a controller communicatively coupled to the pulse generator. The controller is configured to receive a signal and determine whether to cause the pulse generator to apply TID therapy to a patient's heart based at least in part upon the received signal.

### A. FIELD OF THE DISCLOSURE

The present disclosure relates generally to cardiac stimulation systems, and more particularly to an implantable cardiac device that provides temporary induced dyssynchrony therapy.

### B. BACKGROUND ART

Heart failure (HF) is a debilitating, end-stage disease in which abnormal function of the heart leads to inadequate blood flow to fulfill the needs of the body's tissues. Typically, the heart loses propulsive power because the cardiac muscle loses capacity to stretch and contract. Often, the ventricles do not adequately fill with blood between heartbeats, and the valves regulating blood flow may become develop leaks, allowing regurgitation or backflow of blood. The impairment of arterial circulation deprives vital organs of oxygen and nutrients Fatigue, weakness, and inability to carry out daily tasks may result. Not all HF patients suffer debilitating symptoms immediately. Some may live actively for years. Yet, with few exceptions, the disease is relentlessly progressive. As HF progresses, it tends to become increasingly difficult to manage.

Using temporary induced dyssynchrony (TID) therapy to create regular, periodic asynchrony in HF patients without underlying dyssynchrony has been shown to facilitate improving cardiac chamber function, cellular function, and cardiac reserve. Specifically, at least some known TID therapy, such as pacemaker-induced transient asynchrony (PITA) therapy that uses a pacemaker to induce asynchrony, involves using right ventricular (RV) pacing to induce forced ventricular asynchrony in a patient's heart at regular intervals (e.g., for a period of six hours every night for six weeks). One concept behind TID therapy is that the heart may benefit from “exercise” (i.e., forcing the heart into ventricular asynchrony), similar to other muscles in the body.

Since TID pacing is not hemodynamically favorable, it may be uncomfortable or even intolerable for the patient. Therefore, it may be beneficial to determine whether a patient can tolerate TID therapy before applying the TID therapy to the patient.

### BRIEF SUMMARY OF THE DISCLOSURE

In one embodiment, the present disclosure is directed to an implantable cardiac device for providing temporary induced dyssynchrony (TID) therapy. The implantable cardiac device includes a plurality of electrodes, a pulse generator coupled to the electrodes and a controller communicatively coupled to the pulse generator. The controller is configured to receive a signal and determine whether to cause the pulse generator to apply cardiac therapy to a patient's heart via the plurality of electrodes to temporarily induce dyssnchrony based at least in part upon the received signal.

In another embodiment, the present disclosure is directed to an implantable cardiac device that includes a pulse generator coupled to a plurality of electrodes. The computing device includes a memory device, and a processor communicatively coupled to the memory device, the processor configured to receive a signal, and determine whether to cause the pulse generator to apply temporary induced dyssynchrony (TID) therapy to a patient's heart based at least in part upon the received signal.

In another embodiment, the present disclosure is directed to a method for providing temporary induced dyssynchrony (TID) therapy. The method includes receiving a signal, and determining whether to apply TID therapy to a patient's heart based at least in part on the received signal and delivering TID therapy to the patient's heart in response to the determination.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1A is a simplified, partly cutaway view illustrating an implantable stimulation device in electrical communication with at least three leads implanted into a patient's heart for delivering multi-chamber stimulation and shock therapy and sensing cardiac activity.

FIG. 1B is a functional block diagram of the multi-chamber implantable stimulation device of FIG. 1A, illustrating the basic elements that provide pacing stimulation, cardioversion, and defibrillation in four chambers of the heart.

FIG. 2 shows a flowchart of a process for determining whether to apply temporary induced dyssynchrony (TID) therapy.

FIG. 3 shows a flowchart of a process for determining whether to continue ongoing TID therapy.

Corresponding reference characters indicate corresponding parts throughout the several views of the drawings.

### DETAILED DESCRIPTION OF THE DISCLOSURE

The present disclosure describes systems and methods for providing temporary induced dyssynchrony (TID) therapy. An implantable cardiac device includes a plurality of electrodes. A controller communicatively coupled to the plurality of electrodes is configured to receive a signal and determine whether to cause the plurality of electrodes to apply TID therapy to a patient's heart based at least in part upon the received signal.

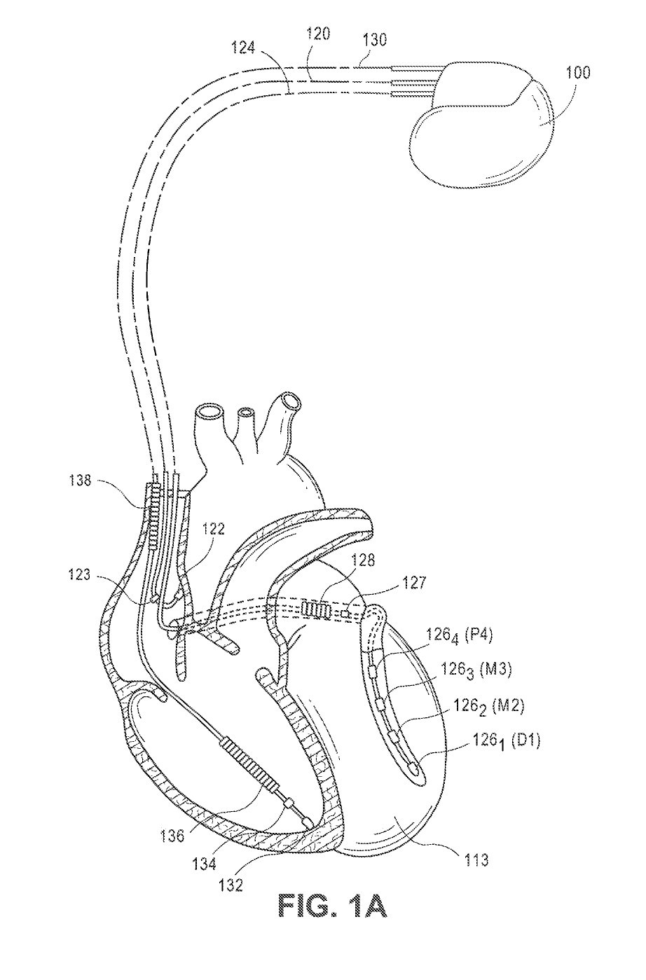

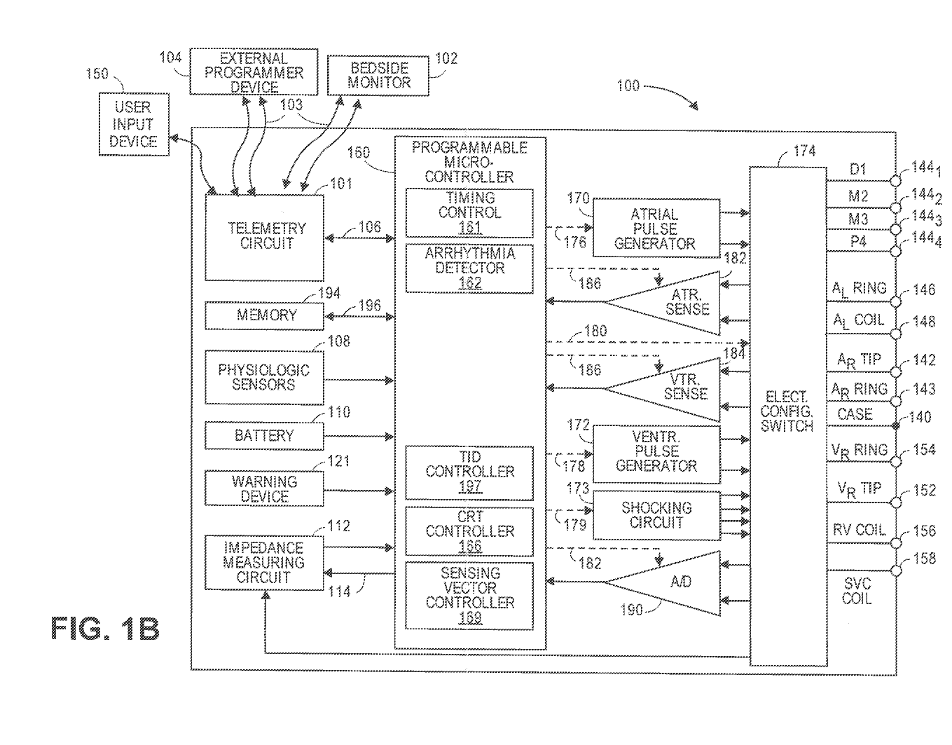

With reference to FIGS. 1A and 1B, a description of an example pacemaker/implantable cardioverter-defibrillator (ICD) 100 will now be provided. FIG. 1A is a simplified block diagram of pacemaker/ICD 100, which is a dual-chamber stimulation device capable of treating both fast and slow arrhythmias with stimulation therapy, including cardioversion, defibrillation, and pacing stimulation, including multipoint pacing (MPP). To provide atrial chamber pacing stimulation and sensing, pacemaker/ICD 100 is shown in electrical communication with a heart 113 by way of a right atrial (RA) lead 120 having an atrial tip electrode 122 and an atrial ring electrode 123 implanted in the atrial appendage. Pacemaker/ICD 100 is also in electrical communication with heart 113 by way of a right ventricular (RV) lead 130 having, in this embodiment, a ventricular tip electrode 132, a RV ring electrode 134, a RV coil electrode 136, and a superior vena cava (SVC) coil electrode 138. Typically, RV lead 130 is transvenously inserted into the heart so as to place RV coil electrode 136 in the RV apex, and SVC coil electrode 138 in the superior vena cava. Accordingly, RV lead 130 is capable of receiving cardiac signals, and delivering stimulation in the form of pacing and shock therapy to the right ventricle (also referred to as the RV chamber).

To sense left atrial and ventricular cardiac signals and to provide left chamber pacing therapy, pacemaker/ICD 100 is coupled to a multi-pole left ventricular (LV) lead 124 designed for placement in the “CS region” for positioning a distal electrode adjacent to the left ventricle and/or additional electrode(s) adjacent to the left atrium (also referred to as the LA chamber). As used herein, the phrase “CS region” refers to the venous vasculature of the left ventricle, including any portion of the coronary sinus (CS), great cardiac vein, left marginal vein, left posterior ventricular vein, middle cardiac vein, small cardiac vein, and/or any other cardiac vein accessible by the CS. Accordingly, an example LV lead 124 is designed to receive atrial and ventricular cardiac signals and to deliver left ventricular pacing therapy using a set of four LV electrodes 126₁, 126₂, 126₃, and 126₄ (thereby providing a quadra-pole lead), left atrial pacing therapy using at least a LA ring electrode 127, and shocking therapy using at least a LA coil electrode 128. In some embodiments, LV lead 124 includes LV electrodes 126₁, 126₂, 126₃, and 126₄, but does not include LA ring and coil electrodes 127 and 128. Such a lead can be, e,g., the Quartet™ left ventricular pacing lead developed by Abbott Laboratories, which includes four pacing electrodes on the left ventricular lead—enabling up to ten pacing configurations

LV electrode 126₁ is shown as being the most “distal” LV electrode (with relation to how far the electrode is from where LV lead 124 connects to pacemaker/ICD 100). For example LV electrode 126₁ may be located at the apex of the left ventricle. LV electrode 126₄ is shown as being the most “proximal” LV electrode. For example LV electrode 126₄ may be located at the base of the left ventricle. LV electrodes 126₂ and 126₃ are shown as being “middle” LV electrodes, between distal and proximal LV electrodes 126₁ and 126₄. Accordingly, the four LV electrodes 126₁, 126₂, 126₃, and 126₄ can be referred to respectively as electrodes D1, M2, M3 and P4 (where “D ” stands for “distal”, “M” stands for “middle”, and “P” stands from “proximal”, and the numbers are arranged from most distal to most proximal). It is also possible that more or fewer LV electrodes are provided. However, for much of the remaining discussion, it will be assumed that the multi-pole LV lead 124 includes four LV electrodes 126₁, 126₂, 126₃, and 126₄ LV electrodes D1, M2, M3 and P4, respectively).

LV electrodes 126₁, 126₂, 126₃, and 126₄ can be used to provide various pacing vectors and sensing vectors. Some of the vectors are intraventricular LV vectors (vectors between two LV electrodes); whereas others are interventricular vectors (e.g., vectors between an LV electrode and RV coil electrode 136). Below is a list of exemplary vectors that can be used for pacing and/or sensing using LV electrodes D1, M2, M3 and P4 with and without the RV coil electrode 136. In the following list, the first electrode in each row (i.e., the electrode to the left of the arrow) is assumed to be connected as the cathode, and the second electrode in each row the electrode to the right of the arrow) is assumed to be connected as the anode, but that need not be the case, especially where neither electrode is a coil.

D1⇄RV coil

M2⇄RV coil

M3⇄RV coil

P4⇄RV coil

D1⇄M2

D1⇄P4

M2⇄P4

M3⇄M2

M3⇄P4

P4⇄M2

Alternative and/or additional vectors, other than those listed above, can be used for pacing and/or sensing. Although only three leads are shown in FIG. 1A, it should also be understood that additional leads (with one or more pacing, sensing, and/or shocking electrodes) might be used and/or additional electrodes might be provided on the leads already shown, such as additional electrodes on the RV or LV lead. It is also possible that less than three leads be used.

A simplified block diagram of internal components of pacemaker/ICD 100 is shown in FIG. 1B, While a particular pacemaker/ICD is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation. A housing 140 for pacemaker/ICD 100, shown schematically in FIG. 1B, is often referred to as the “can”, “case” or “case electrode” and may be programmably selected to act as the return electrode for all “unipolar” modes. Housing 140 may further be used as a return electrode alone or in combination with one or more of coil electrodes, 128, 136 and 138 for shocking purposes. Housing 140 further includes a connector (not shown) having a plurality of terminals, 142, 143, 144₁-144₄, 146, 148, 152, 154, 156 and 158 (shown schematically and, for convenience, the names of the electrodes to which they are connected are shown next to the terminals). As such, to achieve RA sensing and pacing, the connector includes at least an RA tip terminal (A_{R} TIP) 142 adapted for connection to the atrial tip electrode 122 and an RA ring (A_{R} RING) electrode 143 adapted for connection to atrial ring electrode 123. To achieve left chamber sensing, pacing and shocking, the connector includes an LV tip terminal 144₁ adapted for connection to the D1 electrode and additional LV electrode terminals 144₂, 144₃ and 144₄ terminals adapted for connection to the M2, M3 and P4 electrodes of quadra-pole LV lead 124.

The connector also includes an LA ring terminal (A_{L} RING) 146 and an LA shocking terminal (A_{L} COIL) 148, which are adapted for connection to LA ring electrode 127 and the LA coil (A_{L} COIL) electrode 128, respectively. To support right chamber sensing, pacing and shocking, the connector further includes an RV tip terminal (V_{R} TIP) 152, an RV ring terminal (V_{R} RING) 154, an RV shocking terminal (V_{R} COIL) 156, and an SVC shocking terminal (SVC COIL) 158, which are adapted for connection to ventricular tip electrode 132, RV ring electrode 134, RV coil electrode 136, and SVC coil electrode 138, respectively.

At the core of pacemaker/ICD 100 is a programmable microcontroller 160, which controls the various modes of stimulation therapy. As is well known in the art, microcontroller 160 (also referred to herein as a control unit or controller) typically includes a microprocessor, or equivalent control circuitry, designed specifically for controlling the delivery of stimulation therapy and may further include RAM or ROM memory, logic and timing circuitry, state machine circuitry, and I/O circuitry. Typically, microcontroller 160 includes the ability to process or monitor input signals (data) as controlled by a program code stored in a designated block of memory.

As shown in FIG. 1B, an atrial pulse generator 170 (controlled by a control signal 176) and a ventricular pulse generator 172 (controlled by a control signal 178) generate pacing stimulation pulses for delivery by RA lead 120, RV lead 130, and/or LV lead 124 via an electrode configuration switch 174. Microcontroller 160 includes timing control circuitry 161 to control the timing of the stimulation pulses, including, but not limited to, pacing rate, atrio-ventricular (AV) delay, interatrial conduction (AA) delay, interventricular conduction (VV) delay and/or intraventricular delay (e.g., LV1-LV2 delay). Timing control circuitry 161 can also keep track of timing of refractory periods, blanking intervals, noise detection windows, evoked response detection windows, alert intervals, marker channel timing, local time, pacing (e.g., dosage or therapy) duration, non-pacing duration, etc.

Microcontroller 160 further includes an arrhythmia detector 162 that can be utilized by pacermaker/ICD 100 for determining desirable times to administer various therapies. Additional components of the microcontroller may include a cardiac resynchronization therapy (CRT) controller 168 to control CRT and a temporary induced dyssynchrony (TID) controller 197 (described in detail below).

Microcontroller 160 is also shown as including a sensing vector controller 169 that can be used, e,g., to control the electrode configuration switch 174 (e.g., via control signals 180) to selectively connect specific electrode(s) to sensing circuits 182 or 184 as a cathode or an anode, to achieve the various sensing vectors that are used to obtain intracardiac electrograms (IEGMs) in accordance with embodiments described herein. Where multiple sensing vectors are being used to obtain a plurality of IEGMs indicative of cardiac electrical activity at a plurality of ventricular regions, sensing circuit 184 may include multiple channels (e.g., duplicate circuitry) to enable sensing of more than one ventricular IEGM signal at the same time, and/or sensing circuit 184 may use time divisional multiplexing to sense more than one ventricular IEGM signal.

Depending upon the implementation, the various components of the microcontroller may be implemented as separate software modules or the modules may be combined to permit a single module to perform multiple functions. For example, CRT controller 168 and TID controller 197 may be combined. In addition, although shown as being components of the microcontroller, some or all of these components may be implemented separately from the microcontroller, using application specific integrated circuits (ASICs) or the like.

Switch 174 includes a plurality of switches for connecting the desired electrodes to the appropriate I/O circuits, thereby providing complete electrode programmability. Accordingly, switch 174, in response to a control signal 180 from microcontroller 160, determines the polarity of the stimulation pukes (e.g., unipolar, bipolar, combipolar (e.g., using unipolar leads in the atrium and ventricle and performing atrial sensing in a bipolar way using the ventricular lead tip as an indifferent electrode), etc.) by selectively closing the appropriate combination of switches (not shown) as is known in the art. The switch also switches among the various LV electrodes.

Atrial sensing circuits 182 (controlled by a control signal 186) and ventricular sensing circuits 184 (controlled by a control signal 188) may also be selectively coupled to RA lead 120, LV lead 124, and RV lead 130, through switch 174 for detecting the presence of cardiac activity in each of the four chambers of the heart. Accordingly, the atrial (ATR. SENSE) and ventricular (VTR. SENSE) sensing circuits, 182 and 184, may include dedicated sense amplifiers, multiplexed amplifiers or shared amplifiers. Switch 174 determines the “sensing polarity” of the cardiac signal by selectively closing the appropriate switches, as is also known in the art. In this way, a clinician may program the sensing polarity independent of the stimulation polarity. Each sensing circuit, 182 and 184, preferably employs one or more low power, precision amplifiers with programmable gain and/or automatic gain control, bandpass filtering, and a threshold detection circuit, as known in the art, to selectively sense the cardiac signal of interest. The automatic gain control enables pacemaker/ICD 100 to deal effectively with the difficult problem of sensing the low amplitude signal characteristics of atrial or ventricular fibrillation. The outputs of the atrial and ventricular sensing circuits, 182 and 184, are connected to the microcontroller 160 which, in turn, are able to trigger or inhibit the atrial and ventricular pulse generators, 170 and 172, respectively, in a demand fashion in response to the absence or presence of cardiac activity in the appropriate chambers of the heart.

Cardiac signals are applied to the inputs of an analog-to-digital (A/D) data acquisition system 190 (controlled by a control signal 192). Data acquisition system 190 is configured to acquire intracardiac electrogram signals, convert the raw analog data into a digital signal, and store the digital signals for later processing and/or telemetric transmission to an external programmer device 104 or a bedside monitor 102 or personal advisory module. Data acquisition system 190 is coupled to RA lead 120, LV lead 124, and RV lead 130 through switch 174 to sample cardiac signals across any pair of desired electrodes. Microcontroller 160 is further coupled to a memory 194 by a suitable data/address bus 196, wherein the programmable operating parameters used by microcontroller 160 are stored and modified, as required, in order to customize the operation of pacemaker/ICD 100 to suit the needs of a particular patient. Such operating parameters define, for example, the amplitude or magnitude, pulse duration, electrode polarity, for both pacing pulses and impedance detection pulses as well as pacing rate, sensitivity, arrhythmia detection criteria, and the amplitude, waveshape and vector of each pacing and shocking pulse to be delivered to the patient's heart within each respective tier of therapy. Other pacing parameters include base rate, rest rate and circadian base rate.

Advantageously, the operating parameters of implantable pacemaker/1CD 100 may be non-invasively programmed into memory 194 through a telemetry circuit 101 in telemetric communication with external programmer device 104 or bedside monitor 102, such as a programmer, transtelephonic transceiver, or a diagnostic system analyzer. The telemetry circuit 101 is activated by the microcontroller by a control signal 106, Telemetry circuit 101 advantageously allows intracardiac electrograms and status information relating to the operation of pacemaker/ICD 100 (as contained in microcontroller 160 or memory 194) to be sent to external programmer device 104 and/or bedside monitor 102 through an established communication link 103. Additionally, telemetry circuit 101 enable communication between microcontroller 160 and a user input device 150. User input device 150 may include any kind of user computing device, such as a mobile phone, a laptop, a tablet, a wearable computing device (e.g., a fitness wearable or “smart glasses”) or may include any other kind of input device, such as a remote control or an input device specifically configured for communication with pacemaker/ICD 100 to control functionality thereof. User input device 150 may additionally or alternatively include a “smart home controller” or similar Internet of Things device. An internal warning device 121 (also referred to as a patient alert) may be provided for generating perceptible warning signals to the patient via vibration, voltage or other methods.

Pacemaker/ICD 100 further includes and/or is in communication with one or more physiologic sensors 108. Physiologic sensor 108 may include an accelerometer, and may be referred to as a “rate-responsive” sensor because it is typically used to adjust pacing stimulation rate according to the exercise state of the patient. Physiologic sensor 108 may additionally or include a blood pressure sensor, a heart rate sensor, a temperature sensor, an impedance sensor, an activity sensor, and/or a blood oxygenation sensor.

Pacermaker/ICD 100 additionally includes a battery 110 that provides operating power to the circuits shown in FIG. 1B. As further shown in FIG. 1B, pacemaker/ICD 100 is shown as having an impedance measuring circuit 112, which is enabled by the microcontroller 160 via a control signal 114. Uses for an impedance measuring circuit include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring respiration; and detecting the opening of heart valves, etc. Impedance measuring circuit 112 is advantageously coupled to switch 174 so that any desired electrode may be used.

In the case where pacemaker/ICD 100 is intended to operate as an implantable cardioverter/defibrillator (ICD) device, it detects the occurrence of an arrhythmia, and automatically applies an appropriate electrical shock therapy to the heart aimed at terminating the detected arrhythmia. To this end, microcontroller 160 further controls a shocking circuit 173 by way of a control signal 179. Shocking circuit 173 generates shocking pulses of low (up to 0.1 joules), moderate (0.1-10 joules) or high energy (11 to 40 joules or more), as controlled by the microcontroller 160. Such shocking pulses are applied to the heart of the patient through at least two shocking electrodes, and as shown in this embodiment, selected from LA coil electrode 128, RV coil electrode 136, and/or SVC coil electrode 138. Housing 140 may act as an active electrode in combination with RV coil electrode 136, or as part of a split electrical vector using SVC coil electrode 138 or LA coil electrode 128 (i.e., using RV coil electrode 136 as a common electrode).

In this embodiment, microcontroller 160 further includes temporary induced dyssynchrony (TID) controller 197. TID controller 197 controls pacemaker/ICD 100 to apply TID therapy (e.g., pacemaker-induced transient asynchrony (PTA) therapy) to a patient's heart. In particular, TID controller 197 receives a signal and processes that signal to determine hether to apply TID therapy. In the example embodiment, TID controller 197 may be initially programmed to initiate TID therapy at night, when the patient is asleep. However, in some cases, the sensation of initiating TID therapy and/or terminating TID therapy may be noticeable to the patient, even while sleeping or if the patient is not fully asleep. Accordingly, TID controller 197 is configured to use the received signal to determine whether the patient is in a state in which the patient is likely able to tolerate TID therapy (e.g., without waking the patient, or with minimal disruption to the patient's quality of life), such that the patient is more likely to maintain compliance with the TID therapy.

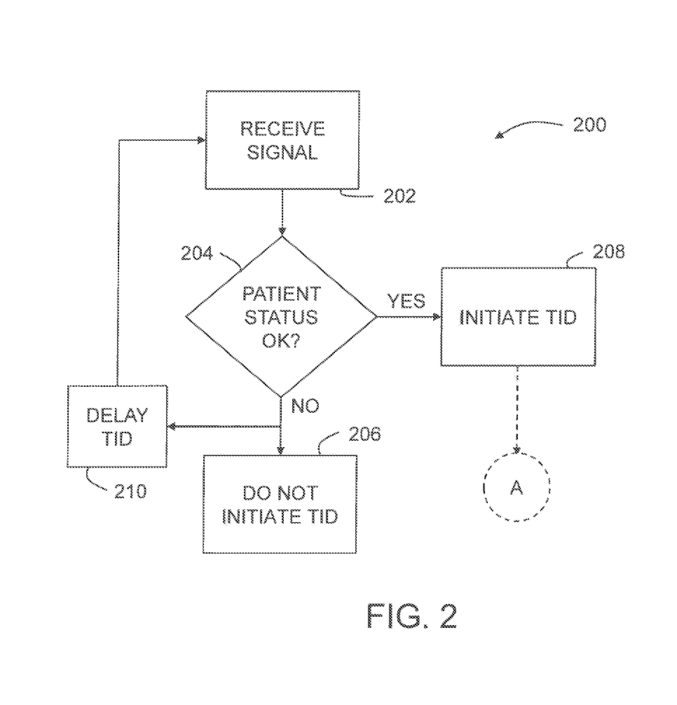

FIG. 2 shows a flowchart of a process 200 for determining whether to apply TID therapy. Process 200 may be implemented, in the example embodiment, using pacemaker/ICD 100 (shown in FIGS. 1A and 1B). More particularly, process 200 may be implemented, at least in part, using TID controller 197 (shown in FIG. 1B). Process 200 is implemented to analyze a status of the patient before TID therapy is initiated. Because TID therapy intentionally stresses the patient's head, it may be desirable to determine whether the patient is in a stable state and can tolerate the induced asynchrony. For example, if a patient is nearing decompensated HF, it may be undesirable to initiate TID therapy.

Process 200 includes receiving 202 a signal. In some embodiments, the signal is received 202 from a sensor (e.g., sensor 108 and/or sensing vector controller 169, shown in FIG. 1B) and includes a patient status indicator. For example, the sensor may be configured to measure and provide a patient status indicator including vital signs or surrogate measures, such as systemic blood pressure, filling pressure, baseline heart rate, heart rate variability, temperature, and/or impedance indicators of tissue edema, congestion, or pulmonary arterial pressure (PAP). The patient status indicator may additionally or alternatively include a sleep or activity status. In another embodiment, the signal is received 202 from a user input device (e.g., user input device 150, shown in FIG. 1B) and includes a user input signal. For example, TID controller 197 may transmit a control signal causing the user input device to display one or more prompts to the patient to enter information. The patient uses the user input device to respond to the prompts, for example, to enter patient status indicators or answer health questions in one particular example, the user input device displays a prompt such as “Can you tolerate TID tonight?”. The patient enters their response, which is received 202 as an input signal. In another particular example, the user input device displays an alert or indicator to the patient that TID therapy is about to be initiated (e.g., according to a schedule). The alert or indicator may include a visual indicator, such as blinking lights, or an audible indicator, such as beeping or vibration of the user input device. In such a case, the patient uses the user input device to confirm the TID therapy or to cancel initiation of the TID therapy.

Process 200 further includes determining 204 whether to initiate TID therapy based at least in part upon the received 202 signal. In one embodiment, TID controller 197 determines 204 whether the patient is in a state in which the patient can tolerate TID therapy based on the received 202 signal. For example, if the received 202 signal includes a patient status indicator that indicates the patient is experiencing pulmonary congestion or that indicates a filling pressure is elevated, TID controller 197 determines 204 that the patient cannot tolerate TID therapy and does not initiate 206 TID therapy. As another example, a signal received 202 from a user input device includes a patient input signal indicating that the patient cannot tolerate TID therapy. Accordingly, TID controller 197 determines 204 that the patient cannot tolerate TID therapy and does not initiate 206 TID therapy. Conversely, if TID controller 197 determines 204 that the patient can tolerate TID therapy, TID controller 197 initiates 208 PITA therapy (e.g., by causing a plurality of electrodes to apply TID therapy to the patient's heart).

1n another case, a signal received 202 from a sensor such as a fitness wearable or sleep tracker may indicate whether a patient is tired, or what sleep state the patient is in. If the patient is tired but not asleep, it may not be appropriate to initiate a scheduled TID session. However, if the patient is in a sufficiently deep sleep (e.g., REM sleep), the patient may be able to tolerate TID therapy without disruption of the patient's sleep. Additionally or alternatively, a signal received 202 from timing control circuitry (e.g., timing control circuitry 161, shown in FIG. 1B) indicates that a change in local time has occurred (e.g., the patient has travelled) and, accordingly, it is not appropriate to initiate a scheduled session of TID therapy. Additionally or alternatively, signal received 202 from a sensor such as a fitness wearable, activity tracker, or sleep tracker may indicate whether a patient is still active at a time when a session of TID therapy is scheduled (e.g., if the patient is out late). TID controller 197 determines 204, based on such received 202 signals, whether the TID therapy may be disruptive to the patient and either initiates 208 TID therapy or does not initiate 206 TID therapy. In one or more alternative embodiments, TID controller 197 may determine 204 that the TID therapy may be disruptive at the current time and according may delay 210 TID therapy for a later time within a predetermined period of time, such as within the same night. When TID controller 197 delays 210 TID therapy, TID controller 197 may continuously and/or periodically receive 202 signals to determine 204 when to initiate 208 TID therapy at a later time.

In some embodiments, process 200 includes additional or alternative steps. For instance, in some embodiments, initiating 208 TID therapy includes varying one or more parameters of TID therapy. TID therapy, such as PITA therapy, uses induced RV pacing to deliver a brief period of asynchrony to the patient's heart. In some embodiments, a sudden initiation of asynchrony can be unpleasant to the patient. Accordingly, TID controller 197 may vary one or more parameters of the TID therapy to gradually introduce the asynchrony. For example, where a shortened atrioventricular (AV) delay introduces the asynchrony, TID controller 197 may cause the ventricular pulse generator 172 to gradually shorten the AV delay over a predetermined period of time (e.g., several minutes). In one embodiment, the AV delay is shorted from 200 ms to 60 ms, by intervals of 20 ms every minute. In such embodiments, the patient's heart will transition from a purely intrinsic rhythm to fusion of intrinsic activity and pacing as the AV delay gradually shortens, and will subsequently transition to a pure pacing rhythm as the AV delay further shortens.

Determining 204 whether to initiate TID therapy includes, in some embodiments, comparison of a received 202 signal to one or more thresholds or ranges. For example, TID controller 197 compares a received 202 patient status indicator of a patient's blood pressure to a threshold blood pressure to determine 204 whether the patient's blood pressure is below a maximum threshold blood pressure. As another example, TID controller 197 compares a received 202 patient status indicator of a patient's heart rate to a range of “acceptable” heart rates to determine 204 whether the patient's heart rate is within an acceptable range to initiate 208 TID therapy. In some embodiments, TID controller 197 automatically determines 204 whether to apply TID therapy based on a received 202 signal being within a threshold range indicating that the patient can tolerate TID therapy. Accordingly, process 200 proceeds to initiating 208 TID therapy.

TID controller 197 is configured to process and analyze various received 202 signals, such as sleep, tiredness, patient willingness, blood pressure, heart rate, congestion, etc., representative of a patient status, in order to determine 204 how to proceed. As described above, TID controller 197 may determine 204 to initiate 208 TID therapy, including initiating 208 “regular” TID therapy and/or gradual-onset TID therapy, delay 210 To therapy, or not initiate 206 TID therapy. Moreover, in some cases, TID controller 197 is configured to proceed with another process 300 for determining whether to continue TID therapy, after TID therapy is initiated 208.

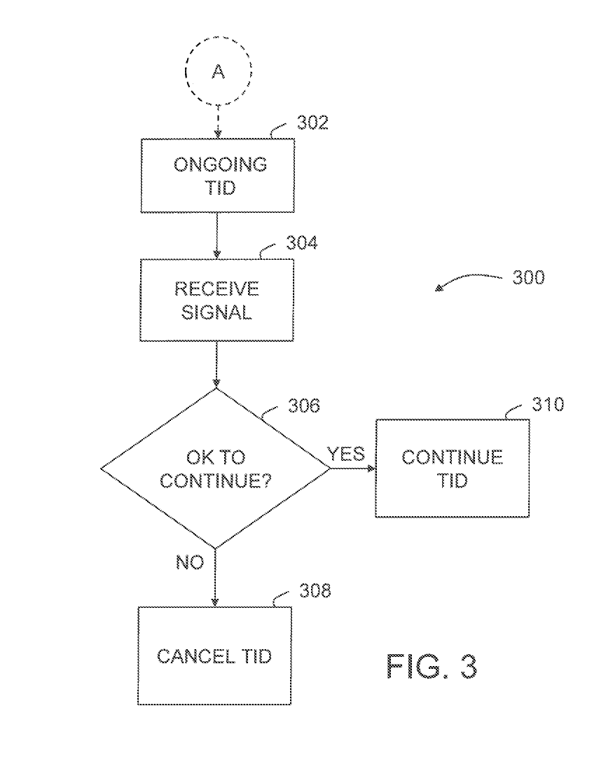

FIG. 3 shows a flowchart of process 300 for determining whether to continue TID therapy. Process 300 may be implemented, in the example embodiment, using pacemaker/ICD 100 (shown in FIGS. 1A and 1B), More particularly, process 300 may be implemented, at least in part, using TID controller 197 (shown in FIG. 1B). Process 300 is implemented to analyze a status of the patient during ongoing TID therapy. Accordingly, process 300 includes applying 302 ongoing TID therapy. In some cases, process 300 is implemented subsequent to process 200 (shown in FIG. 2), such that applying 302 ongoing TID therapy follows initiating 208 TID therapy. Additionally or alternatively, process 300 is implemented independently of process 200.

Process 300 further includes receiving 304 a signal. Receiving 304 a signal may be substantially similar to receiving 202 (shown in FIG. 2). For instance, in some embodiments, the signal is received 304 from a sensor (e.g., sensor 108 and/or sensing vector controller 169, shown in FIG. 1B) and includes a patient status indicator. In another embodiment, the signal is received 304 from a user input device (e.g., user input device 150, shown in FIG. 1B) and includes a user input signal.

Process 300 includes determining 306 whether to continue the ongoing TID therapy. In some embodiments, TID controller 197 automatically determines 306 whether to continue the ongoing TID therapy based on a received 304 signal being within a threshold range indicating that the patient can tolerate TID therapy. If the received 304 signal indicates that the patient status has deteriorated during the ongoing TID therapy, TID controller 197 determines 306 that the patient cannot tolerate the ongoing TID therapy and cancels 308 the TID therapy. As another example, the received 304 signal, such as a patient status indicator of a sleep or activity status from a wearable device or an ocular sensor, may indicate that the patient is having an undesirable reaction to the ongoing TID therapy (e.g., changes in sleep status, physical motion, twitching, blinking pattern, movement of the pupils). In this case, TID controller 197 determines 306 that the patient cannot tolerate the ongoing TID therapy and cancels 308 the TID therapy. Other received 304 signals that could be used as indicators of TID intolerance include changes in respiration, heart rate, heart rate variability, activity, and posture. For instance, if there is a sudden change in a respiratory pattern (e.g., measured by an impedance sensor or a similar sensor), it could indicate that the TID therapy is causing physiologic distress.

In some cases, TID controller 197 cancels 308 ongoing TID therapy if the received 304 signal includes a patient status indicator that falls outside of a threshold range for the patient status indicator. In addition, TID controller 197 continues 310 ongoing TID therapy if the received 304 signal includes a patient status indicator that falls within a threshold range for the patient status indicator. In other words, TID controller 197 continues 310 ongoing TID therapy if the received 304 signal indicates that the patient can tolerate the ongoing TID therapy and/or that the ongoing TID therapy is not disruptive to the patient.

In other cases, TID controller 197 cancels 308 ongoing TID therapy if the received 304 signal includes a patient status indicator that falls outside of a threshold range based on an initially received patient status indicator (e.g., a signal received 202 before PITA therapy is initiated). In other words, TID controller 197 cancels 308 ongoing TID therapy if a variation in the patient status indicator is too high.

It is contemplated that, in some embodiments, process 200 and/or process 300 are implemented multiple times over several “trial” sessions of TID therapy. Instead of starting a patient with a full session of TID therapy (i.e., TID therapy at full intensity over a predetermined period of time), TID controller 197 is programmed to cause delivery of one or more trial sessions of TID therapy (i.e., TID therapy at lower intensity and/or for a shorter period of time). TID controller 197 causes the initiation (e.g., step 208 of process 200) of a first trial session of TID therapy. After the application of the trial session and/or during the trial session, TID controller 197 receives 202/304 a signal including a patient status indicator. TID controller 197 uses the received 202/304 signal to determine 204/306 if the trial session of TID therapy induced any physiologic changes in the patient. If TID controller 197 determines 204/306 that any excessive physiologic changes have occurred (e.g., increased PAP after several seconds to minutes of TID therapy), TID controller 197 may cancel 308 the TID therapy. Additionally or alternatively, TID controller 197 may initiate 208 a reduced trial session of TID therapy for the second trial session (e.g., decrease in intensity or duration of the trial session).

The systems and methods described herein leverage the concept that beneficial effects of TID therapy are recognized when TID therapy is applied for regular, periodic intervals. Acknowledging that in order to better ensure that a patient maintains a TID therapy schedule, it may be beneficial to determine whether initiating or continuing a session of therapy will be disruptive or intolerable to the patient. Accordingly, providing TID therapy only when it is tolerable or non-disruptive may improve patient compliance with TID therapy and facilitate achieving the full beneficial effects in patients' hearts.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense,

What is claimed is:

1. An implantable cardiac device for providing temporary induced dyssynchrony (TID) therapy, the implantable cardiac device comprising:
at least one lead having a plurality of electrodes;
a pulse generator communicatively coupled to the plurality of electrodes; and
a controller configured to receive a signal and determine whether to cause the pulse generator to deliver TID therapy to a patient's heart via the plurality of electrodes based at least in part upon the received signal.

2. The implantable cardiac device of claim 1, wherein the controller is further configured to:
receive the signal before initiating TID therapy; and
determine whether to initiate TID therapy based at least in part upon the received signal,

3. The implantable cardiac device of claim 1, wherein the controller is further configured to:
receive the signal during ongoing TID therapy; and
determine whether to continue the ongoing TID therapy based at least in part upon the received signal.

4. The implantable cardiac device of claim 1, wherein the controller is further configured to:
receive the signal after a trial session of TID therapy; and
determine wherein to apply a full session of TID therapy based at least in part upon the received signal.

5. The implantable cardiac device of claim 1, wherein the received signal includes a patient status indicator.

6. The implantable cardiac device of claim 1 further comprising at least one sensor, wherein the controller is further configured to receive the signal from the at least one sensor.

7. The implantable cardiac device of claim 6, wherein the at least one sensor comprises at least one of a blood pressure sensor, a heart rate sensor, a temperature sensor, an impedance sensor, an activity sensor, an accelerometer, a sleep sensor, and a blood oxygenation sensor.

8. The implantable cardiac device of claim 1, wherein the controller is in communication with a user input device and is further configured to receive the signal from the user input device.

9. The implantable cardiac device of claim 1, wherein the controller is further configured to cause the pulse generator to vary at least one parameter of the TID therapy during application of the TID therapy.

10. The implantable cardiac device of claim 9, wherein the at least one parameter includes an atrioventricular (AV) delay, the controller further configured to cause the pulse generator to gradually shorten the AV delay over a predetermined period of time.

11. The implantable cardiac device of claim 1, wherein the controller comprises timing control circuitry, the controller further configured to:
receive the signal from the timing control circuitry, the received signal including an indication of a current time; and
based at least in part upon the current time, determine whether to initiate the application of TID therapy.

12. An implantable cardiac device that includes a plurality of electrodes, the implantable cardiac device comprising:
a memory device;
a pulse generator; and
a processor communicatively coupled to the memory device and the pulse generator, the processor configured to:
receive a signal; and
determine whether to cause the pulse generator to apply temporary induced dyssynchrony (TID) therapy to a patient's heart based at least in part upon the received signal.

13. The computing device of claim 12, wherein the processor is further configured to:
receive the signal before initiating TID therapy; and
determine whether to initiate TID therapy based at least in part upon the received signal.

14. The computing device of claim 12, wherein the processor is further configured to:
receive the signal during ongoing TID therapy; and
determine whether to continue the ongoing TID therapy based at least in part upon the received signal.

15. The computing device of claim 12 further comprising at least one sensor, wherein the processor is further configured to receive the signal from the at least one sensor.

16. The computing device of claim 15, wherein the at least one sensor comprises at least one of a blood pressure sensor, a heart rate sensor, a temperature sensor, an impedance sensor, an activity sensor, an accelerometer, a sleep sensor, and a blood oxygenation sensor.

17. The computing device of claim 12, wherein the processor is further configured to cause the pulse generator to vary at least one application parameter of the TID therapy during application of the TID therapy.

18. The computing device of claim 17, wherein the at least one application parameter includes an atrioventricular (AV) delay, the processor further configured to cause the pulse generator to gradually shorten the AV delay over a predetermined period of time.

19. A method for providing temporary induced dyssynchrony (TID) therapy, the method comprising:
receiving a signal;
determining whether to apply TID therapy to a patient's heart based at least in part on the received signal; and
applying TID therapy to the patient's heart in response to the determination.

20. The method of claim 19, wherein receiving a signal comprises receiving a signal representative of one or more of blood pressure, a heart rate, a temperature, impedance, activity, sleep state, and a blood oxygenation.
